(19) United States
(12) Patent Application Publication
(10) Pub. No.: US 2005/0059660 A1
(43) Pub. Date: Mar. 17, 2005

(54) Novel combination

(75) Inventors: David Nathan Abraham Fox, Sandwich (GB); Eric Howard Karran, Sandwich (GB)

Correspondence Address: PHARMACIA CORPORATION;GLOBAL PATENT DEPARTMENT, POST OFFICE BOX 1027, ST. LOUIS, MO, 63006, US

(73) Assignee: Pharmacia & Upjohn Company

(21) Appl. No.: 10/902,316

(22) Filed: Jul. 29, 2004

Related U.S. Application Data
(60) Provisional application No. 60/500,748, filed on Sep. 4, 2003.

(30) Foreign Application Priority Data
Aug. 1, 2003 (GB) 0318094.0

(51) Int. Cl. A61K 31/5377; A61K 31/416; A61K 31/198; A61K 31/18

(52) U.S. Cl. 514/232.2; 514/234.5; 514/406; 514/563; 514/602

(57) ABSTRACT

Combinations comprising a) an activator of soluble guanylate cyclase and b) an inhibitor of angiotensin converting enzyme (ACE) are useful for treating hypertension.

Priority is hereby claimed of previously filed foreign applications, UK 0318094.0, filed Aug. 1, 2003 (37 C. F. R. § 1.55(a)), which was filed under the Paris Convention for the Protection of Industrial Property and was filed in the United Kingdom with, and received by The Patent Office, Cardiff Road, Newport, South Wales, NP10 8QQ; and U.S. Provisional Application No. 60/500,748, filed Sep. 4, 2003.

The invention relates to the use of a combination of a) a soluble guanylate cyclase activator (sGCa) and b) an inhibitor of angiotensin converting enzyme (ACE) for treating cardiovascular and metabolic diseases, particularly hypertension.

Blood pressure (BP) is defined by a number of haemodynamic parameters taken either in isolation or in combination. Systolic blood pressure (SBP) is the peak arterial pressure attained as the heart contracts. Diastolic blood pressure is the minimum arterial pressure attained as the heart relaxes. The difference between the SBP and the DBP is defined as the pulse pressure (PP).

Hypertension, or elevated BP, has been defined as a SBP of at least 140 mmHg and/or a DBP of at least 90 mmHg. By this definition, the prevalence of hypertension in developed countries is about 20% of the adult population, rising to about 60-70% of those aged 60 or more, although a significant fraction of these hypertensive subjects have normal BP when this is measured in a non-clinical setting. Some 60% of this older hypertensive population have isolated systolic hypertension (ISH), i.e. they have an elevated SBP and a normal DBP. Hypertension is associated with an increased risk of stroke, myocardial infarction, atrial fibrillation, heart failure, peripheral vascular disease and renal impairment (Fagard, R H ; Am. J. Geriatric Cardiology 11(1), 23-28, 2002; Brown, M J and Haycock, S; Drugs 59(Suppl 2), 1-12, 2000).

The pathophysiology of hypertension is the subject of continuing debate. While it is generally agreed that hypertension is the result of an imbalance between cardiac output and peripheral vascular resistance, and that most hypertensive subjects have abnormal cardiac output and increased peripheral resistance, there is uncertainty which parameter changes first (Beevers, G et al.; BMJ 322, 912-916, 2001).

Despite the large number of drugs available in various pharmacological categories, including diuretics, alpha-adrenergic antagonists, beta-adrenergic antagonists, calcium channel blockers, angiotensin converting enzyme inhibitors and angiotensin receptor antagonists, the need for effective treatments of hypertension is still not satisfied.

ACE inhibitors, which block the vasoconstrictive action of the renin-angiotensin-aldosterone system, are recommended as a first-line therapy for hypertension. They are efficacious and generally considered to be well tolerated. The most common side effect, reported by 10-20% of patients, is coughing. Other less frequently reported side effects include rash, angioedema, hyperkalemia and functional renal failure.

Activators of soluble guanylate cyclase increase intracellular cyclic guanosine monophosphate (cGMP) concentrations resulting in relaxation of the smooth muscle of the vasculature. Soluble guanylate cyclase is pharmacologically activated on binding nitric oxide (NO) at a heme site bound to the protein, and then catalyses the conversion of guanosine triphosphate (GTP) to cGMP. Currently, there are 2 main classes of activators of soluble guanylate cyclase; (a) those that potentiate the actions of NO and require the presence of heme in the Fe^{II} oxidation state (Stasch J-P etal.; Br. J. Pharmacol. 135 (2), 333-343, 2002) and referred to as heme-dependant activators of soluble guanylate cyclase, and (b) those that can activate soluble guanylate cyclase in the absence of the heme or with the heme in the oxidised Fe^{III} form (Stasch J-P et al.; Br. J. Pharmacol. 136 (5), 773-783, 2002) and termed heme-independent activators of soluble guanylate cyclase.

According to a first aspect, the present invention provides the use of a combination comprising a) a soluble guanylate cyclase activator and b) an ACE inhibitor in the manufacture of a medicament for treating diseases, particularly cardiovascular and metabolic diseases, more particularly hypertension.

As used herein, the terms “treating” and “treatment” include palliative, curative and prophylactic treatment. The term “hypertension” includes all diseases characterised by supranormal blood pressure, such as essential hypertension, pulmonary hypertension, secondary hypertension, isolated systolic hypertension, hypertension associated with diabetes, hypertension associated with atherosclerosis, and renovascular hypertension, and further extends to conditions for which elevated blood pressure is a known risk factor. Accordingly, the term “treatment of hypertension” includes the treatment or prevention of complications arising from hypertension, and other associated co-morbidities, including congestive heart failure, angina, stroke, glaucoma and impaired renal function, including renal failure. Metabolic diseases include in particular metabolic syndrome (also known as syndrome X), diabetes and impaired glucose tolerance, including complications thereof, such as diabetic retinopathy and diabetic neuropathy.

Hereinafter combinations of a soluble guanylate cyclase activator and an ACE inhibitor, including combinations of specific soluble guanylate cyclase activators and specific ACE inhibitors, will be referred to as combinations of the invention.

By including agents that act by two different physiological mechanisms it is anticipated that the combinations of the invention will provide antihypertensive medicines with superior properties to those currently available. In particular, the use of an ACE inhibitor should counter the effects of reflex up-regulation of the renin-angiotensin-aldosterone system caused by the hypotensive action of the soluble guanylate cyclase activator. The releasing of this physiological ‘brake’ is expected in a clinical context to result in a synergistic effect of the two agents. This synergy may be a more-than-additive acute effect, or a reduced propensity to the development of tolerance following repeated dosing. Furthermore, it is anticipated that this synergy will allow for a satisfactory clinical outcome to be obtained using a lower total drug exposure, which should reduce the risk of adverse effects, such as orthostatic hypotension, that may be associated with the use of a soluble guanylate cyclase activator alone.

Hereinafter the term “the soluble guanylate cyclase activator” means a soluble guanylate cyclase activator for use in the invention, including all pharmaceutically acceptable salts, solvates and polymorphs of that soluble guanylate cyclase activator. Similarly, the term “the ACE inhibitor” means an ACE inhibitor for use in the invention, including all pharmaceutically acceptable salts, solvates and polymorphs of that ACE inhibitor.

The suitability of the soluble guanylate cyclase activator and the ACE inhibitor can be readily determined by evaluation of their potency and selectivity followed by evaluation of their toxicity, pharmacokinetics (absorption, metabolism, distribution and elimination), etc in accordance with standard pharmaceutical practice. Suitable compounds are those that are potent and selective, have no significant toxic effect at the therapeutic dose, and preferably are bioavailable following oral administration.

Potency for the soluble guanylate cyclase activator can be defined as an EC₅₀ value, being the concentration of compound necessary to increase the enzyme activity in an appropriate assay by 50% of the maximum increase attainable with that compound. EC₅₀ values for the soluble guanylate cyclase activators may be determined using the assay described hereinafter. Preferably, the soluble guanylate cyclase activators have an EC₅₀ of less than 10 μM, more preferably less than 1 μM.

Potency for the ACE inhibitor can be defined as an IC₅₀ value, being the concentration of compound necessary to inhibit the enzyme activity by 50% in a standard assay. Appropriate assays are well known in the art. Many ACE inhibitors are administered as ester prodrugs, and in these cases it is the potency of the active acid that is relevant.

Oral bioavailablity refers to the proportion of an orally administered drug that reaches the systemic circulation. The factors that determine oral bioavailability of a drug are dissolution, membrane permeability and hepatic clearance. Typically, a screening cascade of firstly in vitro and then in vivo techniques is used to determine oral bioavailablity.

Dissolution, the solubilisation of the drug by the aqueous contents of the gastrointestinal tract (GIT), can be predicted from in vitro solubility experiments conducted at appropriate pH to mimic the GIT. Preferably the soluble guanylate cyclase activators have a minimum solubility of 5 μg/ml. Solubility can be determined by standard procedures known in the art such as described in Lipinski C A etal.; Adv. Drug Deliv. Rev. 23(1-3), 3-25, 1997.

Membrane permeability refers to the passage of a compound through the cells of the GIT. Lipophilicity is a key property in predicting this and is determined by in vitro Log D_{7.4} measurements using organic solvents and buffer. Preferably the soluble guanylate cyclase activators have a Log D_{7.4} of −2 to +4, more preferably −1 to +3. The Log D can be determined by standard procedures known in the art such as described in Stopher, D and McClean, S; J. Pharm. Pharmacol. 42(2), 144, 1990.

Cell monolayer assays such as Caco2 add substantially to prediction of favourable membrane permeability in the presence of efflux transporters such as P-glycoprotein, so-called Caco2 flux. Preferably, the soluble guanylate cyclase activators have a Caco2 flux of greater than 2×10⁻⁶ cm.s⁻¹, more preferably greater than 5×10⁻⁶cm.s⁻¹. The Caco2 flux value can be determined by standard procedures known in the art such as described in Artursson, P and Magnusson, C; J. Pharm. Sci, 79(7), 595-600, 1990.

Metabolic stability addresses the ability of the GIT to metabolise compounds during the absorption process or the liver to do so immediately post-absorption: the first pass effect. Assay systems such as microsomes, hepatocytes etc are predictive of metabolic lability. Preferably the soluble guanylate cyclase activators show metabolic stability in the assay system that is commensurate with an hepatic extraction of less then 0.5. Examples of assay systems and data manipulation are described in Obach, R S ; Curr. Opin. Drug Disc. Devel. 4(1), 36-44, 2001 and Shibata, Y etal.; Drug Met. Disp. 28(12), 1518-1523, 2000.

Because of the interplay of the above processes, further support that a drug will be orally bioavailable in humans can be gained by in vivo experiments in animals. Absolute bioavailability is determined in these studies by administering the compound separately or in mixtures by the oral route. For absolute determinations (% orally bioavailable) the intravenous route is also employed. Examples of the assessment of oral bioavailability in animals can be found in Ward, K W et al.; Drug Met. Disp. 29(1), 82-87, 2001; Berman, J et al.; J. Med. Chem. 40(6), 827-829, 1997 and Han, K S and Lee, M G ; Drug Met. Disp. 27(2), 221-226, 1999.

Examples of soluble guanylate cyclase activators for use with the invention are disclosed in: Ko, F N et al.; Blood 84, 4226-4233, 1994; Selwood, D L et al.; J. Med. Chem. 44(1), 78-93, 2001; Straub, A et al.; Bioorg. Med. Chem. Lett. 11, 781-784, 2001; Moreland, R B etal.; J. Urol. 167, S938, 2002; Stasch, J-P et al.; Br. J. Pharmacol. 135(2), 333-343, 2002; Stasch, J-P et aL; Br. J. Pharmacol. 136(5), 773-783, 2002; Straub, A etaL.; Bioorg. Med. Chem. 10, 1711-1717, 2002; Miller, L N etal.; Life Sciences 72(9), 1015-1025, 2003; D E 19744027, EP 0 908 456 (equivalent to U.S. Pat. No. 6,162,819), EP 1 227 099 (equivalent to U.S. Pat. No. 6,518,294), WO98/16223, WO98/16507, WO98/23619, WO99/32460, WO00/02851, WO00/06567, WO00/06568, WO00/06569, WO00/21954, WO00/27394, WO00/31047, WO00/46214, WO00/66582, WO01/19780, WO01/20023, WO01/32604, WO01/83490, WO02/42299, WO02/42300, WO02/42301, WO02/42302, WO02/70459, WO02/70460, WO02/70461, WO02/70462, WO02/70510, WO2/92596 and WO03/04503.

The contents of the published patent applications and journal articles and in particular the general formulae of the therapeutically active compounds of the claims and exemplified compounds therein are incorporated herein in their entirety by reference thereto.

Preferred soluble guanylate cyclase activators for use with the invention include: 3-[2-(4-chlorophenylsulfanyl)phenyl]-N-(4-dimethylaminobutyl)-acrylamide (A-350619, Miller, L N etal.; Life Sciences 72(9), 1015-1025, 2003), 3-(5′-hydroxymethyl-2′-furyl)-1-benzylindazole (YC-1, Ko, F N et. al.; Blood 84, 4226-4233, 1994), 5-chloro-2-[[(5-chloro-2-thienyl)sulfonyl]amino]-N-[4-(4-morpholinosulfonyl)phenyl]benzamide (HMR1766, WO00/02851), 5-cyclopropyl-2-[1-(2-fluorobenzyl)-1H-pyrazolo[3,4-b]pyridin-3-yl]pyrimidin-4-yl-amine (BAY41-2272, Straub, A et al.; Bioorg. Med. Chem. Lett. 11, 781-784, 2001), 2-[1-(2-fluorobenzyl)-1H-pyrazolo[3,4-b]pyridin-3-yl]-5-morpholin-4-yl-pyrimidine-4,6-diamine (BAY41-8543, Stasch, J-P et al.; Br. J. Pharmacol. 135(2), 333-343, 2002) and 4-[((4-carboxybutyl)-{2-[2-(4-(2-phenylethyl)-benzyloxy)phenyl]ethyl}amino)methyl]benzoic acid (BAY58-2667, Stasch, J-P et. al.; B r. J. Pharmacol. 136(5), 773-783, 2002; WO01/19780).

Examples of ACE inhibitors for use with the invention include both direct-acting ACE inhibitors and prodrugs thereof, including alacepril, alindapril, altiopril, benazepril, benazeprilat, captopril, ceronapril, cilazapril, cilazaprilat, delapril, enalapril, enalaprilat, fosinopril, imidapril, indolapril, libenzapril, lisinopril, moexepril, moveltipril, pentopril, perindopril, quinapril, quinaprilat, ramipril, rentiapril, spirapril, temocapril, teprotide, trandolapril and zofenopril. Furthermore, the ACE inhibitor may be a “dual ACE/NEP inhibitor”, i.e. a compound that inhibits both ACE and neutral endopeptidase (NEP), such as, for example, omapatrilat, fasidotril, mixanpril, sampatrilat, gemopatrilat (BMS-189921), MDL-100240 and Z13752A (GW660511).

The pharmaceutical combinations of the invention are useful in the treatment of diseases including cardiovascular and metabolic diseases, and they may also be useful in the treatment of other diseases such as thrombosis, and in the management of patients following percutaneous translumenal coronary angioplasty (“post-PTCA patients”).

Preferably the cardiovascular disorder to be treated is hypertension, congestive heart failure, angina, stroke or renal failure. More preferably the cardiovascular disorder is essential hypertension, pulmonary hypertension, secondary hypertension, isolated systolic hypertension, hypertension associated with diabetes, hypertension associated with atherosclerosis, renovascular hypertension, congestive heart failure, angina, stroke or renal failure. In a particularly preferred embodiment, the disorder to be treated is essential hypertension. In another particularly preferred embodiment, the disorder to be treated is pulmonary hypertension. In another particularly preferred embodiment, the disorder to be treated is secondary hypertension. In another particularly preferred embodiment, the disorder to be treated is isolated systolic hypertension. In another particularly preferred embodiment, the disorder to be treated is hypertension associated with diabetes. In another particularly preferred embodiment, the disorder to be treated is hypertension associated with atherosclerosis. In another particularly preferred embodiment, the disorder to be treated is renovascular hypertension.

Preferably the metabolic disease to be treated is impaired glucose tolerance or diabetes, including complications thereof, such as diabetic retinopathy and diabetic neuropathy. More preferably the metabolic disease is impaired glucose tolerance, type-1 diabetes, non-insulin dependent type-2 diabetes or insulin-dependent type-2 diabetes.

The combination of the invention can be administered alone but will generally be administered in admixture with a suitable pharmaceutical excipient, diluent or carrier selected with regard to the intended route of administration and standard pharmaceutical practice.

For example, the combinations of the invention can be administered orally, buccally or sublingually in the form of tablets, capsules, multi-particulates, gels, films, ovules, elixirs, solutions or suspensions, which may contain flavouring or colouring agents, for immediate-, delayed-, modified-, sustained-, pulsed- or controlled-release applications. The combinations of the invention may also be administered as fast-dispersing or fast-dissolving dosage forms or in the form of a high energy dispersion or as coated particles. Suitable formulations may be in coated or uncoated form, as desired.

Such solid pharmaceutical compositions, for example, tablets, may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

The following formulation examples are illustrative only and are not intended to limit the scope of the invention. Active ingredient means a combination of the invention.

### FORMULATION 1

A tablet is prepared using the following ingredients:
Active ingredient (50 mg) is blended with cellulose (microcrystalline), silicon dioxide, stearic acid (fumed) and the mixture is compressed to form tablets.

### FORMULATION 2

An intravenous formulation may be prepared by combining active ingredient (100 mg) with isotonic saline (1000 ml)

The tablets are manufactured by a standard process, for example, direct compression or a wet or dry granulation process. The tablet cores may be coated with appropriate overcoats.

Solid compositions of a similar type may also be employed as fillers in gelatin or HPMC capsules. Preferred excipients in this regard include lactose, starch, a cellulose, milk sugar or high molecular weight polyethylene glycols. For aqueous suspensions and/or elixirs, the sGCa and ACE inhibitor may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients may be present both within the dosage form i.e. within the matrix, and/or on the dosage form, i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The combinations of the invention can also be administered parenterally, for example, intracavernouslly, intravenously, intra-arterially, intraperitoneally, intrathecally, intraventricularly, intraurethrally, intrasternally, intracranially, intramuscularly or subcutaneously, or they may be administered by infusion or needleless injection techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The following dosage levels and other dosage levels herein are for the average human subject having a weight range of about 65 to 70 kg. The skilled person will readily be able to determine the dosage levels required for a subject whose weight falls outside this range, such as children and the elderly.

The dosage of the combination of the invention in such formulations will depend on its potency, but can be expected to be in the range of from 1 to 500 mg of soluble guanylate cyclase activator and 1 to 100 mg of ACE inhibitor for administration up to three times a day. A preferred dose is in the range 10 to 200 mg (e.g. 10, 25, 50, 100 and 200 mg) of soluble guanylate cyclase activator and 5 to 50 mg (e.g. 5, 10, 25 and 50 mg) of ACE inhibitor which can be administered once, twice or three times a day (preferably once). However the precise dose will be as determined by the prescribing physician and will depend on the age and weight of the subject and severity of the symptoms.

For oral and parenteral administration to human patients, the daily dosage level of a combination of the invention will usually be from to 5 to 500 mg (in single or divided doses).

Thus tablets or capsules may contain from 5 mg to 250 mg (for example 10 to 100 mg) of the combination of the invention for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will appreciate that the combinations of the invention may be taken as a single dose as needed or desired (i.e. prn). It is to be appreciated that all references herein to treatment include acute treatment (taken as required) and chronic treatment (longer term continuous treatment).

The combinations of the invention can also be administered intranasally or by inhalation and are conveniently delivered in the form of a dry powder inhaler or an aerosol spray presentation from a pressurised container, pump, spray, atomiser or nebuliser, with or without the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray, atomiser or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the combinations of the invention and a suitable powder base such as lactose or starch.

Aerosol or dry powder formulations are preferably arranged so that each metered dose or “puff” contains from 1 μg to 50 mg of a combination of the invention for delivery to the patient. The overall daily dose with an aerosol will be in the range of from 1 μg to 50 mg which may be administered in a single dose or, more usually, in divided doses throughout the day.

Alternatively, the combinations of the invention can be administered in the form of a suppository or pessary, or they may be applied topically in the form of a gel, hydrogel, lotion, solution, cream, ointment or dusting powder. The combinations of the invention may also be dermally or transdermally administered, for example, by the use of a skin patch, depot or subcutaneous injection. They may also be administered by the pulmonary or rectal routes.

For application topically to the skin, the combinations of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The combinations of the invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in published international patent applications WO91/11172, WO94/02518 and WO98/55148.

Oral administration of the combinations of the invention is a preferred route, being the most convenient. In circumstances where the recipient suffers from a swallowing disorder or from impairment of drug absorption after oral administration, the drug may be administered parenterally, sublingually or buccally.

The combinations of the invention may be used as part of a triple therapy regimen, i.e. a treatment protocol in which the patient is treated with three pharmaceutical agents. The third agent in the triple therapy may be a second soluble guanylate cyclase activator or ACE inhibitor, or it may be chosen from a third pharmacological group. For example, it may be a neutral endopeptidase inhibitor, an angiotensin 11 receptor antagonist, a phosphodiesterase inhibitor such as sildenafil, a calcium channel blocker such as amlodipine, a statin such as atorvastatin, a beta blocker (i.e. a beta-adrenergic receptor antagonist) or a diuretic.

It will be appreciated that the invention covers the following further aspects and that the embodiments specified hereinabove for the first aspect extend to these aspects:
i) a pharmaceutical combination of the invention (for simultaneous, separate or sequential administration) for treating hypertension;
ii) a kit for treating hypertension, the kit comprising: a) a first pharmaceutical composition comprising a soluble guanylate cyclase activator; b) a second pharmaceutical composition comprising an ACE inhibitor; and c) a container for the compositions;
iii) a method of treating hypertension in a subject comprising treating said patient simultaneously, separately or sequentially with an effective amount of a soluble guanylate cyclase activator and an ACE inhibitor.

As used herein, the term “pharmaceutical combination of the invention” means a combination of the invention (i.e. a combination of a soluble guanylate cyclase activator and an ACE inhibitor) in a pharmaceutically acceptable form, including both single dosage forms and co-presentations of two dosage forms.

Assay

Preferred compounds suitable for use in accordance with the present invention are potent soluble guanylate cyclase activators. In vitro potency can be determined by measurement of their EC₅₀ values (the concentration of compound required for half-maximal activation of enzyme activity).

Human recombinant soluble guanylate cyclase is expressed in either Hi5 or Sf9 insect cells using standard baculovirus expression systems. The sequences of both the α₁ and β₁ subunits of sGC are known (Zabel, U et al.; Biochem J. 335(1), 51-57, 1998). cDNAs to both subunits are prepared as Incyte clones. Recombinant baculovirus is generated using the Bac-to-Bac™ (Invitrogen) or BacVector™ (Novagen) systems in accordance with the manufacturers instructions. Insect cells are co-infected with baculovirus for the two subunits. Cells are then grown in accordance with standard methods. The cells are harvested and lysed, and the recombinant protein is isolated from the lysate by sequential anion exchange chromatography on a Resource™ Q column (Amersham Biosciences) and gel filtration chromatography on a HiLoad™ 26/60 Superdex™ 200 column (Amersham Biosciences). Fractions containing the desired protein are identified by virtue of their absorbance at 431 nm. The protein may optionally be hexahistidine-tagged, in which case the above purification sequence is preceded by a metal chelate chromatography step using a nickel-loaded HiTrap™ metal chelate column (Amersham Biosciences).

Assays are performed in 96 well plates in a total assay volume of 100 μL per well containing 50 mM triethanolamine hydrochloride, 5 mM MgCl₂, 100 μM GTP, 1 μM 3-morpholinosydnonimine (SIN-1), 0.1 μg/mL recombinant human soluble guanylate cyclase, 0.05 mg/mL bovine serum albumin and 3 mM dithiothreitol at pH 7.4. Test compounds are present at varying concentrations and the reaction is started by the addition of substrate. Incubations are performed for 60 minutes at 37° C. and the reactions are terminated by the addition of 2.5 μL of glacial acetic acid and placing the plates on ice. The amount of cGMP formed is quantitated by LCMS (Shimadzu QP8000 fitted with a Hypersil BDS C₁₈ column) in comparison with known standards of cGMP detected at a single mass to charge ratio of 344 (negative ion). The mobile phase isb 0.12% acetic acid (pH 5.0 with ammonia), and 10% methanol. Injection volume is 5 μL.

Animal Study

The efficacy of the combinations of the invention can be demonstrated in an animal model of human hypertension.

Animals

The spontaneously hypertensive rat (SHR) is a widely used model of human hypertension. Male SHRs (20-22 weeks old) are instrumented with Doppler flow probes for the measurement of mesenteric, hindquarters and renal blood flow, aortic blood pressure and heart rate according to published methods (Gardiner, S M etal.; Br. J. Pharmacol. 132(8), 1625-1629, 2001 ).

Drugs

Solutions of ACE inhibitor (1-10 μg/mL), soluble guanylate cyclase activator (10-500 μg/mL) and a combination of ACE inhibitor and soluble guanylate cyclase activator are infused at a rate of 0.4 mL/h throughout the experimental period. Control animals receive compound vehicle.

Protocol

Baseline haemodynamic parameters are recorded. Animals are randomised then treated with the drug solution by continuous infusion over 4 days. Changes in haemodynamic parameters are monitored during the study period for 7 h on each day.

1. The use of a combination of an activator of soluble guanylate cyclase and an inhibitor of angiotensin converting enzyme (ACE) for the preparation of a medicament for the palliative, curative or prophylactic treatment of a cardiovascular or metabolic disorder.

2. The use according to claim 1, wherein the activator of soluble guanylate cyclase has an EC₅₀ value of less than 10 μM in an in vitro assay.

3. The use according to claim 2, wherein the activator of soluble guanylate cyclase has an EC₅₀ value of less than 1 μM in an in vitro assay.

4. The use according to claim 1, wherein the activator of soluble guanylate cyclase is selected from:
3-[2-(4-chlorophenylsulfanyl)phenyl]-N-(4-dimethylaminobutyl)acrylamide (A-350619);
3-(5′-hydroxymethyl-2′-furyl)-1-benzylindazole (YC-1):
5-chloro-2-[[(5-chloro-2-thienyl)sulfonyl]amino]-N-[4-(4-morpholinosulfonyl)-phenyl]benzamide (HMR1766);
5-cyclopropyl-2-[1-(2-fluorobenzyl)-1H-pyrazolo[3,4-b]pyridin-3-yl]pyrimidin-4-ylamine (BAY41-2272);
2-[1-(2-fluorobenzyl)-1H-pyrazolo[3,4-b]pyridin-3-yl]-5-morpholin-4-yl-pyrimidine-4,6-diamine (BAY41-8543) and 4-[((4-carboxybutyl)-{2-[2-(4-(2-phenylethyl)benzyloxy)phenyl]ethyl}amino)-methyl]benzoic acid (BAY58-2667)
and pharmaceutically acceptable salts thereof.

5. The use according to any claim 2, wherein the activator of soluble guanylate cyclase is selected from:
3-[2-(4-chlorophenylsulfanyl)phenyl]-N-(4-dimethylaminobutyl)acrylamide (A-350619);
3-(5′-hydroxymethyl-2′-furyl)-1-benzylindazole (YC-1):
5-chloro-2-[[(5-chloro-2-thienyl)sulfonyl]amino]-N-[4-(4-morpholinosulfonyl)-phenyl]benzamide (HMR1766);
5-cyclopropyl-2-[1-(2-fluorobenzyl)-1H-pyrazolo[3,4-b]pyridin-3-yl]pyrimidin-4-ylamine (BAY41-2272);
2-[1-(2-fluorobenzyl)-1H-pyrazolo[3,4-b]pyridin-3-yl]-5-morpholin-4-yl-pyrimidine-4,6-diamine (BAY41-8543) and
4-[((4-carboxybutyl)-{2-[2-(4-(2-phenylethyl)benzyloxy)phenyl]ethyl}amino)-methyl]benzoic acid (BAY58-2667)
and pharmaceutically acceptable salts thereof.

6. The use according to claim 3, wherein the activator of soluble guanylate cyclase is selected from:
3-[2-(4-chlorophenylsulfanyl)phenyl]-N-(4-dimethylaminobutyl)acrylamide (A-350619);
3-(5′-hydroxymethyl-2′-furyl)-1-benzylindazole (YC-1):
5-chloro-2-[[(5-chloro-2-thienyl)sulfonyl]amino]-N-[4-(4-morpholinosulfonyl)-phenyl]benzamide (HMR1766);
5-cyclopropyl-2-[1-(2-fluorobenzyl)-1H-pyrazolo[3,4-b]pyridin-3-yl]pyrimidin-4-ylamine (BAY41-2272);
2-[1-(2-fluorobenzyl)-1H-pyrazolo[3,4-b]pyridin-3-yl]-5-morpholin-4-yl-pyrimidine-4,6-diamine (BAY41-8543) and
4-[((4-carboxybutyl)-{2-[2-(4-(2-phenylethyl)benzyloxy)phenyl]ethyl}amino)-methyl]benzoic acid (BAY58-2667)
and pharmaceutically acceptable salts thereof.

7. The use according to any preceding claim, wherein the inhibitor of ACE is selected from benazepril, captopril, cilazepril, enalapril, enalaprilat, fosinopril, lisinopril, moexepril, perindopril, quinapril, ramipril and trandolapril and pharmaceutically acceptable salts thereof.

8. The use according to claim 1, wherein the medicament is for the treatment of hypertension, congestive heart failure, angina, stroke, diabetes and impaired glucose tolerance.

9. The use according to claim 6, wherein the medicament is for the treatment of hypertension.

10. The use according to claim 7, wherein the medicament is for the treatment of essential hypertension, pulmonary hypertension, secondary hypertension, isolated systolic hypertension, hypertension associated with diabetes, hypertension associated with atherosclerosis or renovascular hypertension.

11. A pharmaceutical composition comprising an activator of soluble guanylate cyclase and an inhibitor of angiotensin converting enzyme (ACE).

12. A pharmaceutical combination for simultaneous, separate or sequential administration for treating hypertension, comprising an activator of soluble guanylate cyclase and an inhibitor of angiotensin converting enzyme (ACE).

13. A composition comprising an activator of soluble guanylate cyclase and an inhibitor of angiotensin converting enzyme (ACE) for use as a medicament.

14. A kit for treating hypertension, the kit comprising: a) a first pharmaceutical composition comprising a soluble guanylate cyclase activator; b) a second pharmaceutical composition comprising an ACE inhibitor; and c) a container for the compositions.

15. A method of treating hypertension in a subject comprising treating said patient simultaneously, separately or sequentially with an effective amount of an activator of soluble guanylate cyclase and an inhibitor of ACE.
